(19) United States
(12) Patent Application Publication
(10) Pub. No.: US 2011/0054263 A1
(43) Pub. Date: Mar. 3, 2011

(54) Replaceable LED illumination assembly for medical instruments

(76) Inventors: Jim-Son Chou, Irvine, CA (US); Cheng-Hsi Miao, San Jose, CA (US)

(21) Appl. No.: 12/807,092

(22) Filed: Aug. 28, 2010

Related U.S. Application Data
(60) Provisional application No. 61/275,383, filed on Aug. 28, 2009.

(51) Int. Cl. A61B 1/06

(52) U.S. Cl. 600/249

(57) ABSTRACT

A replaceable LED assembly for medical instruments and medical devices including at least one LED(light emitting diode) to be used with the medical instruments that are using incandescent lamps or xenon lamps as illumination source. This LED illumination assembly will also allow exchange of LED lamp's without the use of heat, solder, mechanical tools or physical force beyond the use of hand pressure.

### CROSS-REFERENCE TO RELATED APPLICATIONS

This application claims the benefit of provisional patent applications with Ser. No. 61/275,383 filed 2009 Aug. 28, by the present inventors.

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates generally to a LED illumination assembly that is suitable for the medical instruments and in particularly this invention relates to replacing the LED lamp in the illumination assembly of the medical instruments.

2. Prior Arts

Physicians employ illumination assembly in medical instruments and devices to observe, record, take measurement and assist surgeries. Theses illumination assembly was used in both surgical and diagnostic microscope's, including but not limited to, for example, the slit lamp, the specular microscope, and the fundus camera, and various endoscopes that delivered the light via optical fiber into patients' body. Currently, Incandescent lamps, including halogen added Incandescent type incandescent lamps, and Xenon lamps are two major light sources for illumination of medical instruments and devices. The light emitted by incandescent lamps and the light emitted by Xenon lamps have very different spectrums. Incandescent lamps produce mostly long wavelength red radiation and Xenon flash lamps produce shorter wavelength blue-green radiation. Because of the differences in the spectrums, the reflection and scattering patterns of these two light sources are different. In clinical environment, physicians found that illuminated with different spectrum of lights revealed more information of the tissue health. It is therefore desirable for a light source that can be easily switch from one spectrum to another spectrum without complicate electrical, mechanical and optical arrangement in the medical instruments or devices.

Light emitting diode(LED) is a solid state light source that provides low energy consumption, spectrum range and temporal properties that were not attainable by the Incandescent lamp and/or Xenon lamps. But implementing LED into medical instruments and medical devices requires different design innovation that must accommodate all the unique features of the solid state devices. For example, LED can not function at temperatures higher than 75° C. while Incandescent lamps would be most stable at higher temperatures. Also, because LED has different radiation pattern from the incandescent and Xenon lamps There was no illumination assembly of LED in the medical instruments and medical devices that can accommodate these requirements and the need to change the LED's for different spectrum output or for the replacement. Our invention to accommodate all the unique features of the LED illumination is described in the following embodiment.

In many typical mounting arrangements, LEDs are mounted in a mounting assembly or mount which is then soldered to a printed circuit board(PCB) using reflow surface mount techniques. In such arrangements, to remove and replace a defective or burnt out LED, or to change one LED for another, it is necessary to heat the solder holding the original LED mount in place to its melting point and then remove the original mount, clean the board, and then to resolder a replacement LED mount in its place. Alternatively, a whole new replacement board may be utilized to avoid the steps of replacing LED mount completely. Both of these approaches have their drawbacks with respect to ease of replacement, cost or the like.

In an alternative approach, an LED has been mounted in a thread sleeve which fits in a traditional incandescent light bulb socket. While such an arrangement may have the benefit of being easy to replace in a manner intuitively obvious to the average consumers, it suffers from having a relatively bulky form factor that may prevent optimal design of a lighting fixture to take advantage of the small size of the LED light source. It also has a relatively costly.

Additionally, multiple LED fixtures are becoming more prevalent. These fixtures do not typically have a sufficiently easy and cost effective mechanism for replacing LED's.

### OBJECTS AND ADVANTAGES

It is therefore a primary object of the present application is to overcome the above noted deficiency or the prior arts.

It is another primary object of the present application to develop a LED assembly that can replace known medical instrument that utilizing incandescent lamps or Xenon lamps for illumination.

### SUMMARY OF THE INVENTION

In accordance with the present invention, a LED illumination assembly can be used in medical instrument to replace the incandescent lamps and xenon lamps.

In one innovative aspect, the present innovation relates to the use of releasable and removable LED lamp in the illumination assembly.

In another innovative aspect, the present innovation relates to use of human hand, instead of soldering and electrical power, to replace LED in a medical instrument with a power supply.

In another innovative aspect, the present innovation relates to the use of different LED lamp in the same medical instrument without interfere with the electrical, mechanical and optical environment of the medical instrument.

### BRIEF DESCRIPTION OF THE DRAWINGS

The nature and mode of operation of the present invention will now be more fully described in the following detailed description of the invention taken with the accompanying drawing figures, in which:

FIG. 1 illustrates the side view of LED illumination assembly.

FIG. 2 illustrates another three dimensional view LED illumination assembly.

### DETAILED DESCRIPTION

In the description that follows, like parts are indicated throughout the specification and drawings with the same reference numerals. The present invention is not limited to the specific embodiments illustrated herein.

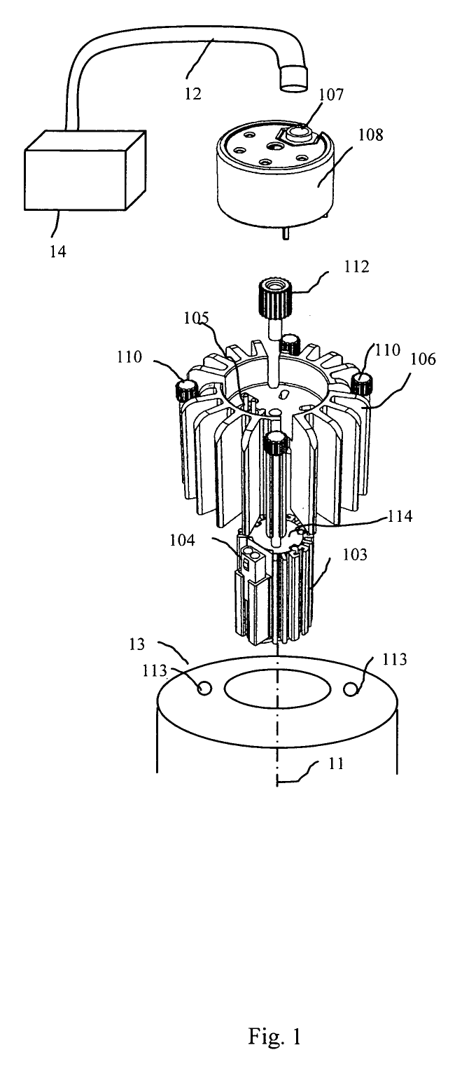

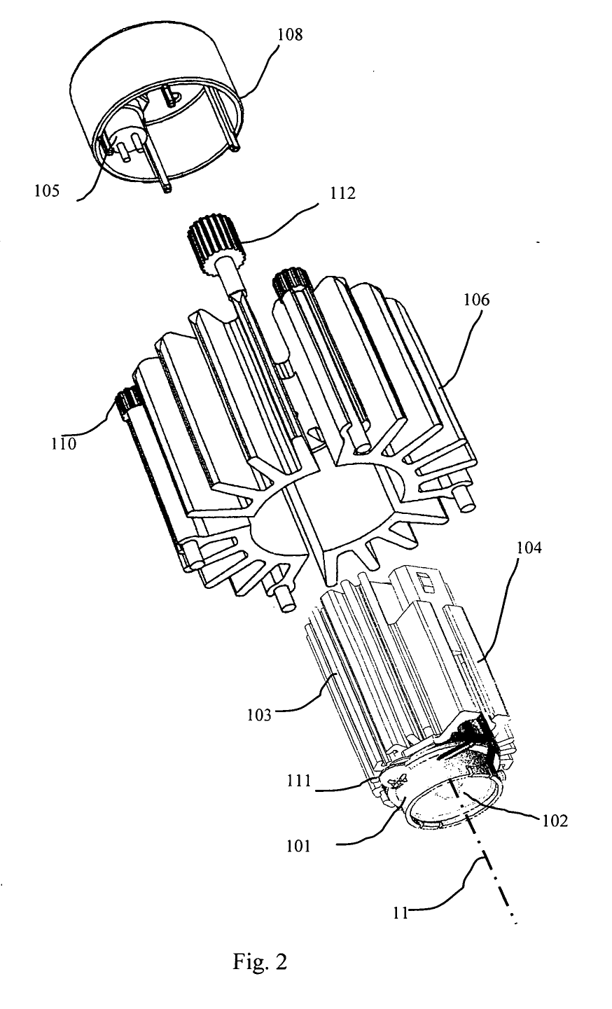

FIG. 1 and FIG. 2 show a schematic view of an embodiment according to the present invention. Referring to FIG. 1 and FIG. 2, the light from the light emitting diode (LED) or LED array 102 is projected along projection axis 11 through projection optical element 101 onto the light beam shaping components of the medical instrument. In one embodiment, projection optical element 101 may comprise a single optical element. In other embodiment, projection optical element 101 may comprise multiple optical elements such as a compound lens or other type of optical elements, including but not limited to the diffractive type of element. In one embodiment, such optical element includes but not limited to the diffusers. In another embodiment, such optical element includes but not limited to the optical filters to select certain wavelength of the light. Projection optics is well known to one skilled in the art and does not require an explanation. In one embodiment, LED 102 contains at least one LED. In another embodiment, LED array 102 is an LED light pattern generator. In one embodiment, the LED or LED array will emit a portion of the visible light spectra with limited bandwidth. In other embodiments, the LED or LED array will emit a white light. The color of the white light can be further controlled by adjusting the electrical power delivered into each individual LED's in the LED array arrangement. This LED or LED array 102 is fastened to the Light Module or LED lamp 103 via fasten elements 111. In current embodiment, the fasten elements 111 are a set of screws. The electrical component of the LED lamp 103 is designed with connector component 104. The LED lamp 103 also provides the heat dissipation structures that will incorporate into the major heat dissipation component 105. In current embodiment, the main heat dissipation component 105, has multiple heat transfer configurations, 106, including but not limited to at least two heat conducting fins are incorporated to facilitate the heat transfer. In one embodiment, the heat dissipation component can also be the housing of the illumination assembly.

In our preferred embodiment, the electricity is delivered to an electrical connector, not shown in the drawings, on the heat dissipation component 105 and eventually to the connector 104 on the LED lamp 103 via connector 107 that is located on the cap 108. The locations of connectors on the heat dissipation component 105 and LED lamp 103 are configured in a way that the fastening of LED lamp 103 onto the heat dissipation component 105 can enforce the connectivity of the electrical connectors. The assembled units 106 and 103 may be attached to the main body of the medical instrument or medical device 13 via a mechanical attachment design using a set of at least two screw holes 113 and a set of at least two hand-turning screws 110. The LED lamp 103 and main heat sink element 105 are attached via attachment element 112, including but not limited to screws that can be turned by hand. In our preferred embodiment, the LED lamp 103 has a hole with screw groove, on the other side of the LED 102, that matches the screw 112 so the LED lamp 103 will be compressed to the surface of the heat dissipation component 105 to reduce the heat impedance between the surfaces of LED lamp 103 and heat dissipation component 105. In our preferred embodiment, such screw 112 can be turned with hand and the electrical connector 104 on the LED lamp will connect to the electrical connector 107 on the heat dissipation component about the same time.

In one embodiment, thermal pad, 114, may be inserted between the surfaces of the LED lamp 103 and the heat sink element 105. In other embodiment, thermal pad, 114 may be omitted between the surfaces of the LED lamp 103 and the heat sink element 105.

In operation, light emitting diode (LED) controller 14 may control LED array 102. In one embodiment, the LED controller 14 may control the LED array to emit light at different intensity. In another embodiment, the LED controller 14 may control the LED array to emit light at different color temperature and at different intensity.

There are numerous variations in the embodiments. Example of these variations include but not limited to, LED lamp 103 may have one or more LEDs; LED array 102 and LED module 103 may have LED of different colors. The patterns of the multiple LED array 102 include but not limited to the LED patterns with no LED element or elements in the center of the pattern. There may be one set of LEDs for emitting a relatively low first light level on LED array 102 or LED module 103, and another set of LEDs for emitting at high light levels. There may be light filters or color filter on the projection axis for changing the spectrum property of the light emitted from illumination assembly 2; the light pattern may change with the light level emitted by the LED light pattern generator using neutral density filters.

Although the embodiment of the invention has been illustrated and that the form has been described, it is readily apparent to those skilled in the art that various modifications may be made therein without departing from the spirit of the invention.

What is claimed is:

1. An illumination assembly suitable for a medical instrument for observing a target tissue comprising:
a releasable and removable LED lamp that includes at least one LED, said LED lamp is able to be fastened to and removed from a heat dissipation component;
an electrical conduction path to deliver the electricity from said instrument to the LED lamp while the LED lamp is assembled to a heat dissipation component or the body of said medical instrument; and
a heat dissipation component that dissipates the heat from said LED lamp while said LED lamp is assembled to said heat dissipation component or the body of said medical instrument; and
a mechanical adapter to accommodate said illumination assembly to the optical design and components that are used to condition the beam of said medical instrument.

2. An illumination assembly according to claim 1, wherein said LED comprising a groups of LED array's.

3. An illumination assembly according to claim 2 wherein said LED array illuminates white lights.

4. An illumination assembly according to claim 2 wherein said LED array illuminates lights of certain wavelength range of the light spectrum.

5. An illumination assembly according to claim 1 wherein said electrical conduction path comprises at least two electrical connectors with one on said LED lamp and the other on said heat dissipation component to maintain the polarity of the electricity and to energize at least one LED or at least one LED array on said LED lamp, while said LED lamp is assembled to said heat dissipation component or the body of said medical instrument.

6. An illumination assembly according to claim 1 wherein said LED lamp comprises at least one hole with screw groove on the back of said LED lamp so said LED lamp may be fastened to said heat dissipation component using a corresponding screw that can be turned with hand while the electrical connector on said LED lamp can be connected to the electrical connector on said heat dissipation component.

7. An illumination assembly according to claim 1 wherein said LED lamp comprises at least one hole with screw groove on the back of said LED lamp so said LED lamp may be fastened to the body of said medical instrument using a corresponding screw that can be turned with hand while the electrical connector on said LED lamp can be attached to the electrical connector on the body of said medical instrument.

8. An illumination assembly according to claim 1 wherein said heat dissipation component comprising a group of metallic heat conducting and heat transfer elements.

9. An illumination assembly according to claim 1 wherein said heat dissipation component comprising a group of non-metallic heat conducting and heat transfer elements includes but not limited to graphite elements and ceramics elements containing Aluminum Oxide(Al₂O₃) and GaN.

10. An illumination assembly according to claim 1 wherein said heat dissipation component comprising a group of heat management elements that facilitates the heat transfer includes but not limited to at least two heat dissipation fins with desired space between them to maximize thermal energy transfer.

11. An illumination assembly according to claim 1 wherein said heat dissipation component includes at least part of the housing of said illumination assembly.

12. An illumination assembly according to claim 1 wherein said adapter comprises a group of mechanical interface that interconnect said heat dissipation component and the main body of the said medical instrument.

13. An illumination assembly according to claim 1 wherein said adapter comprises a group of optical elements of lens, filter and diffusers, that conditions the light for the use of the said medical instrument.

14. An illumination assembly according to claim 1 wherein said LED lamp comprises at least two LED's so different color temperatures can be obtained by adjusting the electrical power that supplies to each LED of said LED lamp.

15. An illumination assembly according to claim 1 wherein said LED lamp comprises at least one thermal pad between the thermal interfaces of said LED lamp and said heating dissipation component, wherein the thermal pad is configured to minimize the thermal impedance between said LED lamp and said heating dissipation component's.

16. A method for adapting a medical instrument so as to incorporate at least one releasable and removable LED lamp as an illumination source, said instrument including a power supply to energize the LED lamp and an optical system that is used to condition the illumination light, said method comprises:
detaching a releasable and removable housing of the illumination assembly;
removing said LED lamp from said instrument assembly;
mounting a desirable said LED lamp to said illumination assembly, including means for electrically interconnecting at least one LED and said power supply as well as means for mechanically and optically interconnecting said medical instrument without the use of heat, solder, mechanical tools or physical force beyond the use of hand pressure.

17. A method according to claim 16, said method including the use of a hand-turning screw to fasten desired said LED lamp onto said heat dissipation component and inserting the electrical connector on said LED lamp to the electrical connector on said heat dissipation component, in the same time, without the use of heat, solder, mechanical tools or physical force beyond the use of hand pressure.

18. A method according to claim 16, said method including unscrewing of a hand-turning screw on said LED lamp from said heat dissipation component and disconnecting the electrical connector on said LED lamp from the electrical connector on said heat dissipation component, in the same time, to remove said LED lamp from said heat dissipation component without the use of heat, solder, mechanical tools or physical force beyond the use of hand pressure.

19. A method according to claim 16, said method including the use of a hand-turning screw to fasten desired said LED lamp to the housing of said medical instrument and inserting the electrical connector on said LED lamp to the electrical connector on the housing of said medical instrument, in the same time, without the use of heat, solder, mechanical tools or physical force beyond the use of hand pressure.

20. A method according to claim 16, said method including unscrewing of a hand-turning screw on said LED lamp from the housing of said medical instrument and disconnecting the electrical connector on said LED lamp from the electrical connector on the housing of said medical instrument, in the same time, to remove said LED lamp from the housing of said medical instrument without the use of heat, solder, mechanical tools or physical force beyond the use of hand pressure.
